(19) United States
(12) Patent Application Publication
(10) Pub. No.: US 2021/0195980 A1
(43) Pub. Date: Jul. 1, 2021

(54) Modular Respiratory Protective System

(71) Applicant: Gentex Corporation, Simpson, PA (US)

(72) Inventors: Richard Cartwright, Hemel Hempstead, Herts (GB); Steven Bridges, Letchworth Garden City, Herts (GB); Chris Stamp, Letchworth Garden City, Herts (GB)

(73) Assignee: Gentex Corporation, Simpson, PA (US)

(21) Appl. No.: 16/754,287

(22) PCT Filed: Oct. 15, 2018
(86) PCT No.: PCT/US2018/055831

§ 371 Date: Apr. 7, 2020

Related U.S. Application Data
(60) Provisional application No. 62/572,976, filed on Oct. 16, 2017.

(51) Int. Cl. A42B 3/28; A61F 9/06; A61F 11/14; A42B 3/22

(52) U.S. Cl. CPC A42B 3/288; A61F 9/068; A42B 3/286; A42B 3/225; A61F 11/14

(57) ABSTRACT

A modular respiratory protection system comprises a frame defining an opening configured to expose a portion of a user's head. A blower is configured to draw air through an air filter and through a portion of the frame. A head protection element is releasably coupled to the frame and the head protection element is configured to cover the opening.

### CROSS-REFERENCE TO RELATED APPLICATIONS

This application claims the benefit of U.S. Provisional Patent Application No. 62/572,976 filed Oct. 16, 2017 entitled “Protection System”, which is incorporated by reference herein in its entirety.

### BACKGROUND OF THE INVENTION

The present invention generally relates to a respiratory protection system and, more particularly, to a modular respiratory protection system that may be configured to protect a user's head, eyes, ears, and/or respiratory system.

### BRIEF SUMMARY OF THE INVENTION

In one embodiment there is a modular respiratory protection system comprising a frame defining an opening configured to expose a portion of a user's head, an air filter, a blower configured to draw air through the air filter and through a portion of the frame, and a head protection element releasably coupled to the frame. The head protection element may cover the opening. The blower is configured to be above a user's ears when the frame is on the user's head. The protections system may include a face shield moveably coupled to the frame.

In a further embodiment, the modular respiratory protection system may include a coupling configured to rotatably couple the face shield to the frame. The coupling may be configured to at least temporarily maintain the face shield in a raised position. The coupling may include a detainer and an intermediate member. The intermediate member may be coupled to the face shield and may be moveable relative to the detainer. The detainer may be configured to resiliently deflect as the intermediate member moves relative to the detainer. The intermediate member may include an arm and the detainer may include a detent. The detent may be configured to at least temporarily prevent movement of the arm. The arm may be configured to cause the detainer to resiliently deflect as the face shield is moved between the raised position and the lowered position and the arm contacts the detent. The face shield may be rotatable between a raised position and a lowered position and the blower may be activated when the face shield is moved from the raised position to the lowered position.

In a further embodiment, the modular respiratory protection system includes an indicator configured to alert a user to a system status, wherein the indicator may be within a user's line of sight when the frame is on the user's head. In a further embodiment, the modular respiratory protection system includes a battery coupled to the frame, the battery being configured to supply power to the blower and the battery being configured to be decoupled from the frame while the frame is on the user's head. The battery may be configured to be decoupled from the frame by the user with one hand. The frame may be configured to be coupled to a face shield and ear muffs. The frame may be configured to be selectively coupled to at least one of a face shield, ear muffs, and a welding mask. The frame may be configured to be interchangeably coupled to a face shield and a welding mask. The welding mask may include an auto-darkening lens. The modular respiratory protection system may include a sensor configured to sense a condition and send a signal to an actuator configured to darken the auto-darkening lens. The frame may be configured to be coupled to an ear muff. The frame may be configured to be coupled to a hood. The head protection element may comprise a bump cap or a hard hat. The air filter may be configured to wrap around a portion of the user's head.

In a further embodiment, the modular respiratory protection system includes a frame defining an opening configured to expose a portion of a user's head, a face shield moveably coupled to the frame, and a coupling configured to rotatably couple the face shield to the frame. The coupling may include a detainer and an intermediate member. The intermediate member may be coupled to the face shield and may be moveable relative to the detainer. The detainer may be configured to resiliently deflect as the intermediate member moves relative to the detainer. The modular respiratory protection system may include an air filter, a blower configured to draw air through the air filter and through a portion of the frame and deliver the air between the face shield and the user's head. The modular respiratory protection system may include a head protection element releasably coupled to the frame and configured to cover the opening.

In a further embodiment, the modular respiratory protection system includes a rigid frame defining an opening configured to expose a portion of a user's head, a face shield moveably coupled to the frame, and an air filter configured to wrap around a portion of the user's head. The modular respiratory protection system may include a blower configured to draw air through the air filter and through a portion of the frame. The blower may be configured to be above a user's ears when the frame is on the user's head. The modular respiratory protection system may include a head protection element releasably coupled to the frame and the head protection element may be configured to cover the opening. The modular respiratory protection system may include a battery coupled to the frame and the battery may be configured to supply power to the blower. The battery may be configured to be decoupled from the frame while the frame is on the user's head.

### BRIEF DESCRIPTION OF THE SEVERAL VIEWS OF THE DRAWINGS

The following detailed description of embodiments of the modular respiratory protection system will be better understood when read in conjunction with the appended drawings of an exemplary embodiment. It should be understood, however, that the invention is not limited to the precise arrangements and instrumentalities shown. For example, although not expressly stated herein, features of one or more various disclosed embodiments may be incorporated into other of the disclosed embodiments.

In the drawings:

FIG. 1 is a front perspective view of a modular respiratory protection system in accordance with an exemplary embodiment of the present invention;

FIG. 2 is a rear perspective view of the modular respiratory protection system of FIG. 1;

FIG. 3 is a top plan view of the modular respiratory protection system of FIG. 1;

FIG. 4 is a left-side elevational view of the modular respiratory protection system of FIG. 1 with the blower cover removed;

FIG. 5 is perspective view of the modular respiratory protection system of FIG. 1 with the face shield in a raised position;

FIG. 6 is an isolated, perspective view of the frame of FIG. 1;

FIG. 7 is a close-up view of a portion of the frame of FIG. 1;

FIG. 8 is an isolated, perspective view of the face shield of FIG. 1;

FIG. 9 is an exploded view of the face shield coupling of FIG. 1;

FIG. 10 is a partially exploded view of the face shield coupling of FIG. 1;

FIG. 11 is a sectional, left side view of the modular respiratory protection system of FIG. 1;

FIG. 12 is an isolated, perspective view of the filter and filter cover of FIG. 1;

FIG. 13 is a rear perspective view of the modular respiratory protection system of FIG. 1 with the insert removed;

FIG. 14 is a rear perspective view of the modular respiratory protection system of FIG. 1;

FIG. 15 is a rear perspective view of the modular respiratory protection system of FIG. 1 with a hood coupled thereto;

FIG. 16 is a right-side perspective view of the modular respiratory protection system of FIG. 1;

FIG. 17 is a right-side perspective view of the modular respiratory protection system of FIG. 1 with the battery removed;

FIG. 18 is a left-side perspective view of the modular respiratory protection system of FIG. 1 with ear muffs coupled thereto;

FIG. 19 is a left-side perspective view of a hard hat in accordance with an exemplary embodiment of the present invention;

FIG. 20 is a left-side perspective view of a bump cap in accordance with an exemplary embodiment of the present invention;

FIG. 21 is a bottom view of the modular respiratory protection system of FIG. 1 with the bump cap attached thereto;

FIG. 22 is a perspective view of the modular respiratory protection system of FIG. 1 with the hard hat attached thereto;

FIG. 23 is a front elevational view of the modular respiratory protection system of FIG. 1 with a welding mask attached thereto;

FIG. 24 is a perspective view of the modular respiratory protection system of FIG. 1 with the welding mask and bump cap attached thereto;

FIG. 25 is a perspective view of the modular respiratory protection system of FIG. 1 with the welding mask and hard had attached thereto;

FIG. 26 is a right-side persepective view of the modular respiratory protection system of FIG. 1 with the welding mask and the ear muffs attached thereto;

FIG. 27 is a perspective view of the modular respiratory protection system of FIG. 1 with the face shield and the hood attached thereto;

FIG. 28 is an isolated, exploded view of the suspension system, band, and frame of FIG. 1;

FIG. 29 is an isolated view of the suspension system, band, and frame of FIG. 1;

FIG. 30 is a front elevational view of the intermediate member of FIG. 1; and

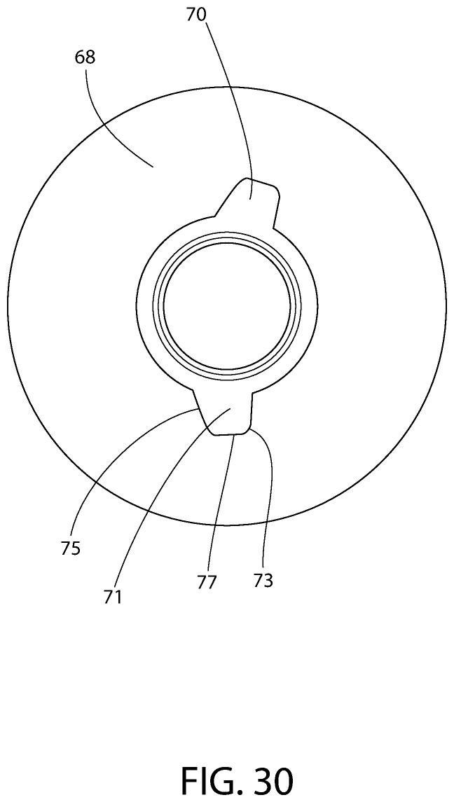

FIG. 31 is an isolated view of the detainer of FIG. 1.

### DETAILED DESCRIPTION OF THE INVENTION

Referring to the drawings in detail, wherein like reference numerals indicate like elements throughout, there is shown a modular respiratory protection system, generally designated 20, in accordance with exemplary embodiments of the present invention. In one embodiment, the modular respiratory protection system 20 (also referred to herein as protection system 20) may be selectively configured to provide protection for at least one of a wearer's face, eyes, ears, and/or head. The protection system 20 may be configured to selectively provide protection against fumes, sound, impact, bumps, fluids, dust, and/or debris. In some embodiments, the protection system 20 includes a filter and a blower as part of a powered air-purifying respirator (PAPR) system. In some embodiments, the protection system 20 includes a supplied air respirator (SAR).

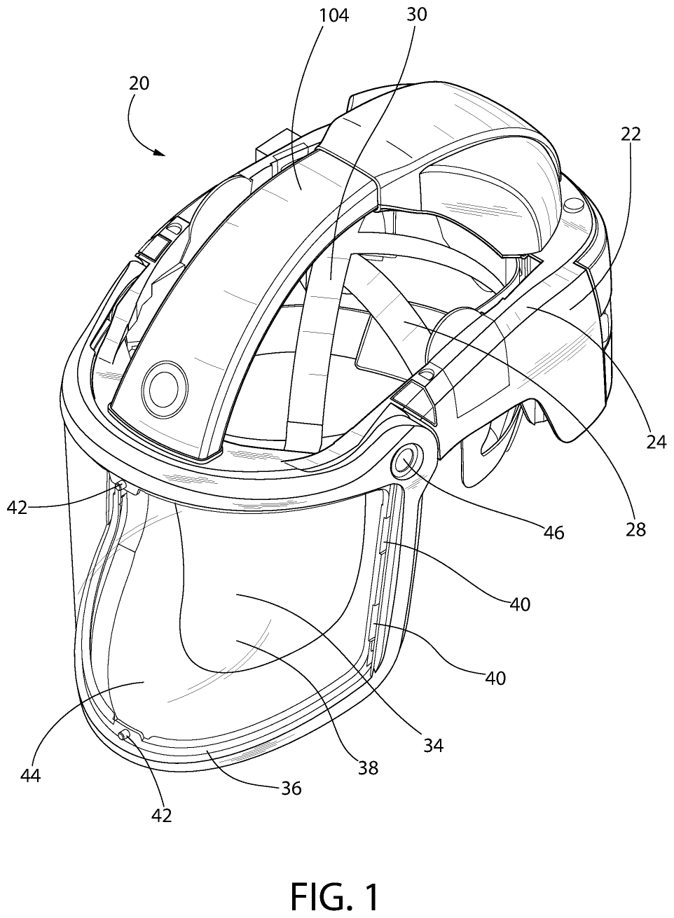

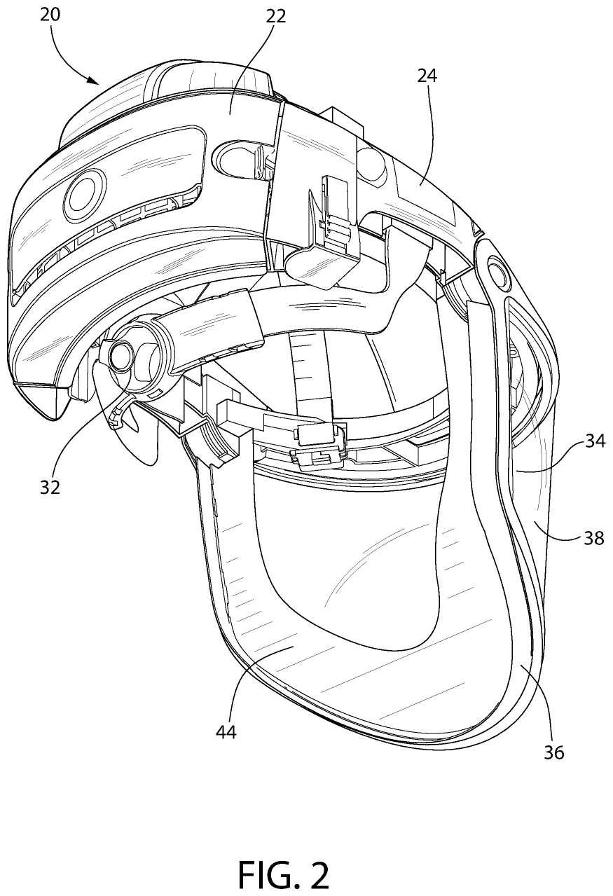

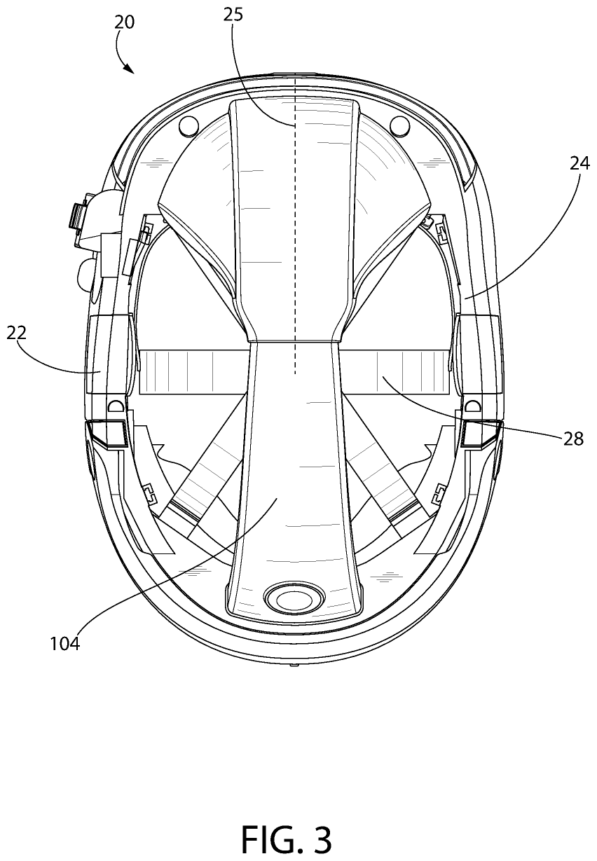

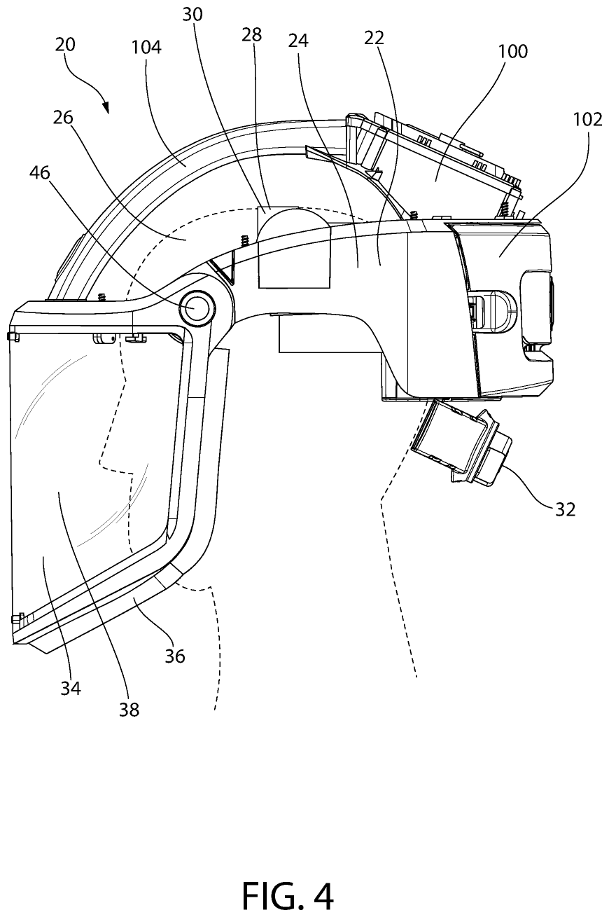

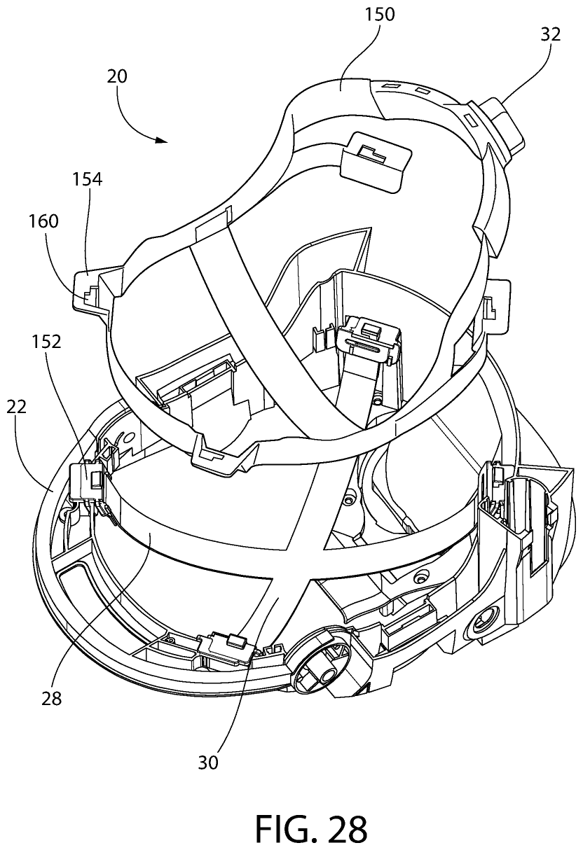

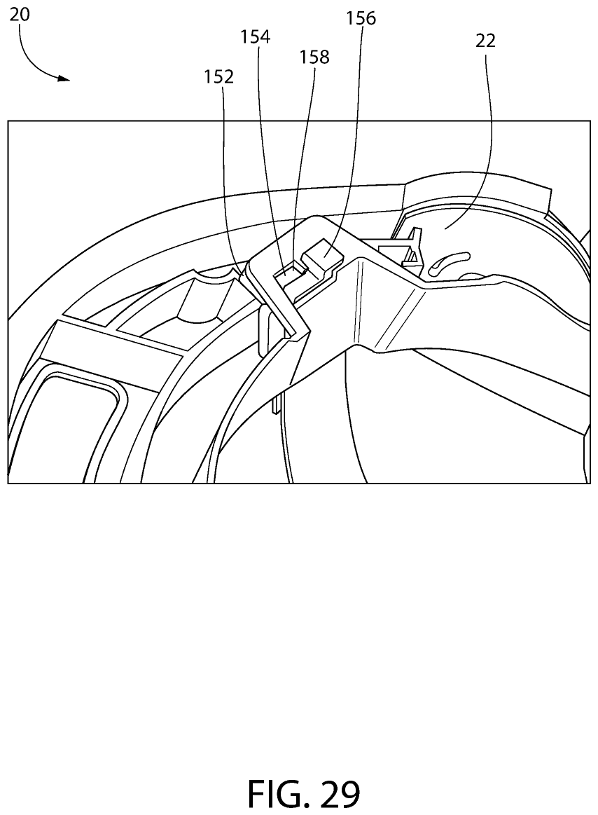

Referring to FIGS. 1-5, the protection system 20 may include a frame 22. The frame 22 may include connection features configured to be coupled to selected elements (e.g., a face shield, a filter and blower, a welding mask, a hood, and/or ear muffs), as explained in greater detail below. The frame 22 may include a halo 24 configured to extend around at least a portion of a user's head 26 (FIG. 3). The frame 22 may be coupled to a suspension system 28 which may be configured to engage the user's head 26. The suspension system 28 may include one or more straps 30. The straps 30 may be flexible such that the suspension system 28 generally secures the frame 22 to the user's head 26 but allows some relative movement between the frame 22 and the user's head 26. Flexible straps may help reduce the force of any impacts on the user's head 26 from the frame 22 when the frame 22 contacts an external element. The suspension system 28 may include an adjustment mechanism 32 (FIG. 4) configured to adjust the size of the suspension system 28 to securely fit on a user's head 26. The adjustment mechanism 32 may be a ratchet system, belt buckle, worm dial, or cinch strap. In some embodiments, in place of or in addition to the suspension system 28, the protection system 20 includes one or more pads for comfort and/or impact absorption. The suspension system 28 may be coupled to the frame 22 by a clip 152 (FIGS. 28-29).

Referring to FIGS. 28-29, a band 150 may be coupled to the suspension system 28. The adjustment mechanism 32 may be coupled to the band 150. The band 150 may be coupled to the suspension system 28 (e.g., the clip 152) by a receiver 154. The clip 152 may be coupled to the frame 22 by a snap-fit connection. The clip 152 may include a tab 156 and a body 158. At least a portion of the tab 156 may be spaced from the body 158. The receiver may include an opening 160 having a first portion and a second portion. The first portion may be larger than the second portion. The band 150 may be coupled to the suspension system 28 by moving the receiver 154 relative to the clip 152 in a first direction such that the tab 156 moves through the first portion of the opening 160. The receiver 154 may be moved in a second direction to move the tab 156 from the first portion to the second portion of the opening 160. The first direction may be different than the second direction. The tab 156 may engage a portion of the receiver 154 when the tab 156 is within the second portion of the opening 160.

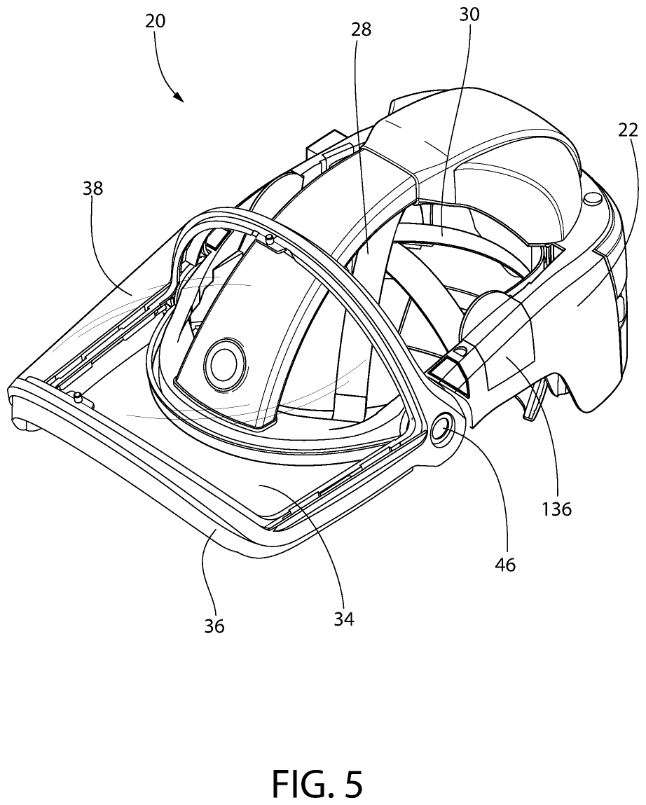

Referring to FIGS. 1-5, the protection system 20 may include a face shield 34. The face shield 34 may be configured to prevent dirt, debris, fluid, and/or other materials from contacting the user's face when the face shield 34 is in the lowered position. The face shield 34 may be moveable relative to the frame 22 from a lowered position (FIG. 1) to a raised position (FIG. 5), as explained in greater detail below. The face shield 34 may include face shield frame 36 and a barrier 38. The face shield frame 36 may be configured to removably receive one of a plurality of barriers 38. The face shield frame 36 may include tabs 40 that define a slot configured to receive a portion of the barrier 38 to secure the barrier 38 to the face shield frame 36. The face shield frame 36 may include a pin 42 configured to be inserted into an aperture on the barrier 38 to secure the barrier 38 to the face shield frame 36. The barrier 38 may be selected based on the environmental hazards that a user will encounter during use of the protection system 20. The barrier 38 may be a solid material configured to prevent the passage of gas, fluid, dirt, and/or debris. The barrier 38 may be a tinted or polarized glass or plastic that provides vision protection against bright lights or glare. The barrier 38 may include a first portion comprising a first barrier (e.g., glass, plastic, or tinted glass) and a second portion comprising a second barrier (e.g., clear glass or plastic). In some embodiments, a user may replace barrier 38 without removing the protection system 20 from their head.

The face shield 34 may include a shroud 44 configured to engage a user's face or neck to prevent material, gas, smoke, or fluid from passing behind the face shield 34 into contact with the user's face. The face shield 34 and shroud 44 may assist the containment of positive air pressure within, while also allowing exhaled air to exit the space between the user's face and the face shield 34. In some embodiments, positive pressure within the space between the user's face and the face shield 34 prevents particles, vapors, gases, or contaminants from entering the space. The shroud 44 may be manufactured from a material comprising polyurethane film with nylon weave backing. In one embodiment, the shroud 44 comprises a thermoplastic elastomer extrusion that is sewn around the outer edge of a face seal and is pressed into a channel in the face shield frame 36. The manufacturing material of the shroud 44 may be selected depending on the environmental hazards a user will encounter during use of the protection system. The shroud 44 may be flexible or elastic such that the shroud 44 adheres to the contours of a user's face to provide a gas or fluid seal with the user's face.

In some embodiments, the face shield 34 is moveable relative to the frame 22. In other embodiments, the face shield 34 is fixed relative to the frame 22. Referring to FIGS. 1 and 5, the face shield 34 may be moveably coupled to the frame 22 such that the face shield 34 can move between the lowered position (FIG. 1) and the raised position (FIG. 5). In one embodiment, the face shield 34 is translated (e.g., laterally or vertically) relative to the frame 22. In one embodiment, the face shield 34 is flexible and folds or is rolled up about itself to move the face shield 34 from the lowered position to a raised position. In some embodiments, the face shield 34 is moveable to a semi-retracted position such that a selected portion (e.g., the user's eyes), but less than all of the user's face, are covered by the face shield 34. In one embodiment, the face shield 34 is rotatable relative to the frame 22.

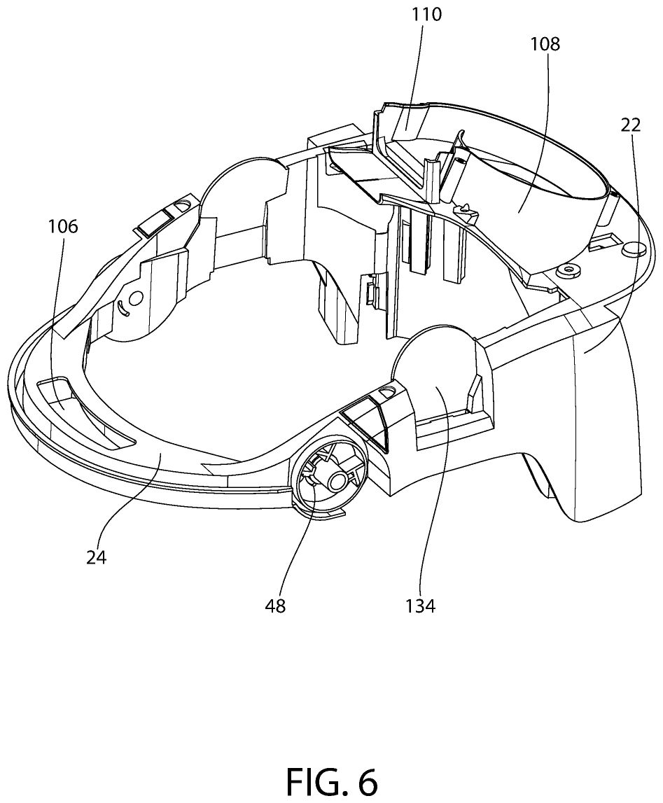

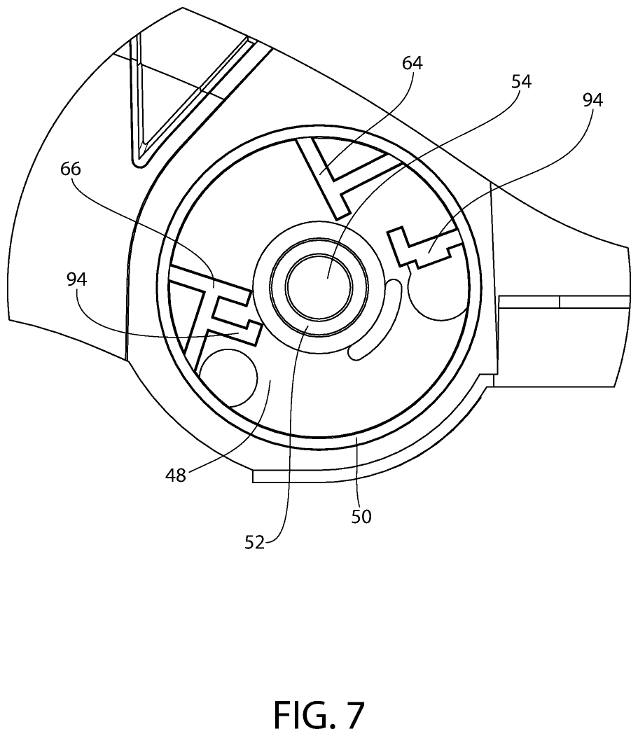

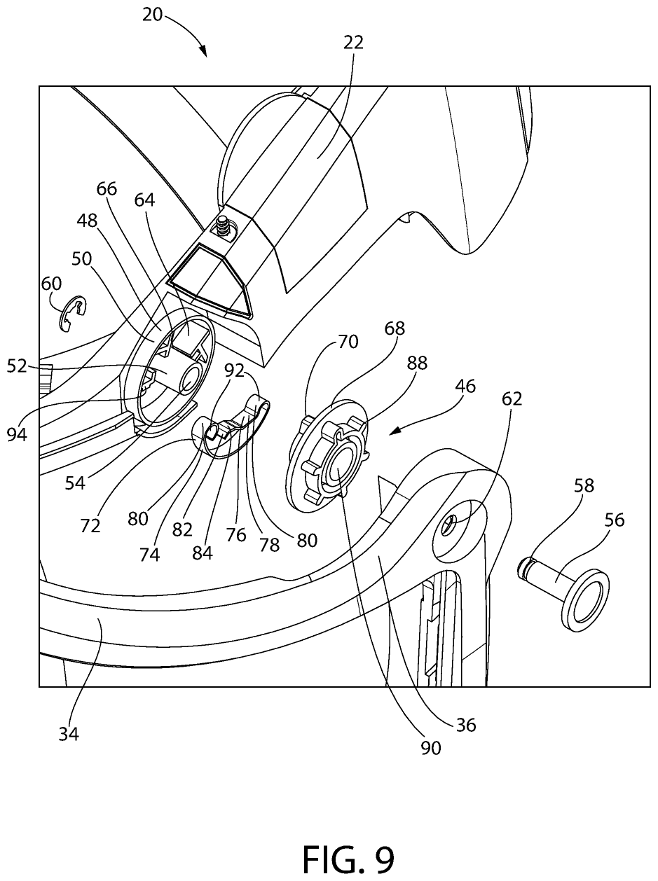

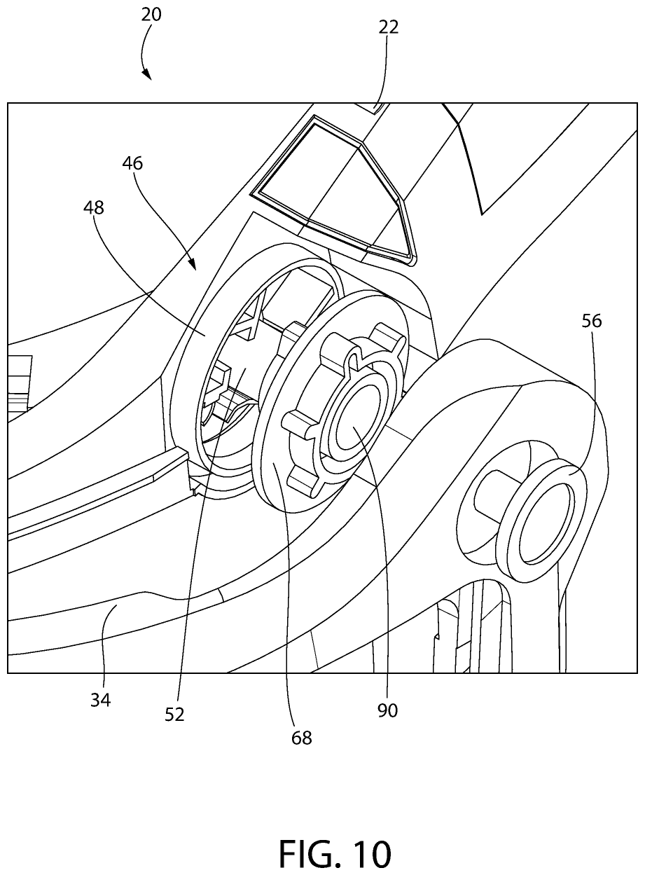

A coupling 46 may be coupled to the frame 22 and the face shield 34 to allow relative rotation between the face shield 34 and the frame 22. The coupling 46 may at least temporarily hold the face shield 34 in a selected one of a plurality of positions relative to the frame 22. Referring to FIGS. 6-10, the coupling 46 may include a receiver 48 fixed to the frame 22. The receiver 48 may include an outer wall 50 (FIG. 7). The outer wall 50 may prevent dirt or debris from entering the coupling 46. The receiver 48 may include a sleeve 52 defining an internal opening 54. The internal opening 54 may be configured to receive a pin 56 (FIGS. 9-10). The pin 56 may include a track 58 adapted to engage a retainer 60 (e.g., retaining ring, hitch pin, or cotter pin) such that the face shield 34 is rotatably coupled to the frame 22 when the pin 56 is inserted through the sleeve 52 and through an opening 62 in the face shield 34. The pin 56 may be detachably coupled to each of the frame 22 and the face shield 34 such that the face shield 34 may be detached from the frame 22. The receiver 48 may include first stop 64 and a second stop 66 (FIG. 7). At least one of the first stop 64 and the second stop 66 may be coupled to the sleeve 52 and the outer wall 50. The receiver 48 may include an abutment 94 spaced from the outer wall 50. The abutment 94 may be coupled to the first stop 64 or the second stop 66. The abutment 94 may be spaced from the first stop 64 and the second stop 66.

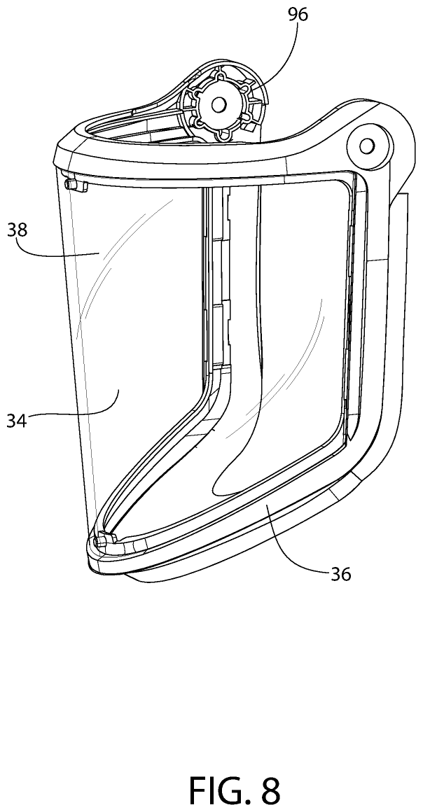

Referring to FIGS. 8-10, the coupling 46 may include an intermediate member 68. The intermediate member 68 may include one or more spokes 88 configured to engage a recess 96 on the face shield 34 (FIG. 8) such that the intermediate member 68 is rotationally fixed relative to the face shield 34. The intermediate member 68 may include an opening 90 configured to receive the sleeve 52 (FIG. 10). The intermediate member 68 may include an arm 70 (FIG. 9) configured to engage the first stop 64 and the second stop 66 to limit rotation of the coupling 46. The first stop 64 may define a stop position for the arm 70 when the face shield 34 is in the raised position. The second stop 66 may define a stop position for the arm 70 when the face shield 34 is in the lowered position.

Referring to FIGS. 9-10 and 31, the coupling 46 may include a detainer 72. The detainer 72 may be configured to be positioned between the outer wall 50 of the receiver 48 and the sleeve 52 (FIG. 10). The detainer 72 may include an outer wall 74 and an inner wall 76. One or more ends 80 may form the transition between the inner wall 76 and the outer wall 74. Each of the ends 80 may define an aperture 92 configured to receive the abutment 94 of the receiver 48 such that the detainer 72 is rotationally fixed relative to the receiver 48. In one embodiment, the inner wall 76 is coupled to one of the apertures 92 and is spaced from another aperture 92. In one embodiment, the inner wall 76 is cantilevered from one of the apertures 92 such that the inner wall 76 can flex between a first position and a second position. In other embodiments, the inner wall 76 and outer wall 74 are formed as a single continuous element.

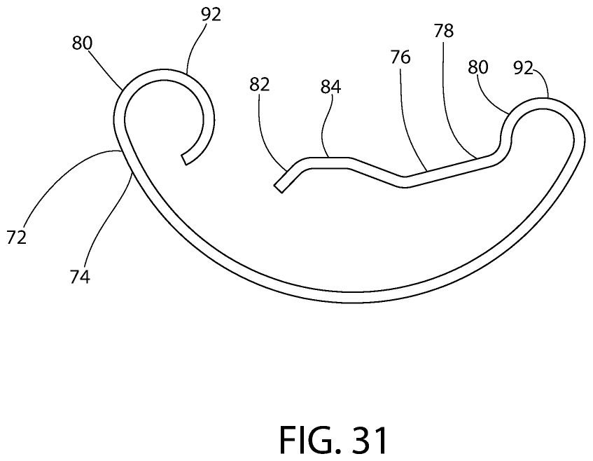

The inner wall 76 may define a first receiving area 78 and a second receiving area 82. Each of the first receiving area 78 and the second receiving area 82 are configured to receive a second arm 71 of the intermediate member 68 (FIG. 31). The second arm 71 may include a first sidewall 73 and a second sidewall 75. In one embodiment, the first sidewall 73 and the second sidewall 75 are disposed at different angles relative to a face 77 of the second arm 71 such that the force required to move the second arm 71 from the first receiving area 78 to the second receiving area 82 is different than the force required to move the second arm 71 from the second receiving area 82 to the first receiving area 78. In some embodiments, the force required to move the shield 34 from the lowered position to the raised position is less than the force required to move the shield 34 from the raised position to the lowered position.

With continued reference to FIGS. 9-10 and 31, a detent 84 may separate the first receiving area 78 from the second receiving area 82. The detent 84 may be a raised portion compared to at least one of the first receiving area 78 and the second receiving area 82. The detent 84 may resist movement of the second arm between the first receiving area 78 and the second receiving area 82 such that the face shield 34 remains in the raised position or lowered position until the user moves the face shield 34. The detainer 72 may be flexible such that inner wall 76 flexes as the face shield 34 is rotated relative to the frame 22 and the second arm moves over the detent 84 as it moves between the first receiving area 78 and the second receiving area 82. The detainer 72 may include more than one detent 84 and more than two receiving areas such that the face shield 34 can be stayed in intermediate positions between the raised position and the lowered position. The detainer 72 may be manufactured from spring steel. The material used to manufacture the detainer 72 may be selected based on the weight of the face shield 34 (e.g., a welding mask may weigh more than a visor).

In one embodiment, the intermediate member 68 is coupled to the face shield 34 and the detainer 72 is rotatably coupled to the intermediate member 68 such that the selected face shield includes an appropriate detainer 72 for the weight of the face shield 34 to maintain the position of the face shield 34 relative to the frame 22. Thus, the frame 22 may be rotatably coupled to a plurality of face shields 34 of different weights without having to replace the detainer 72 each time a different face shield is used. In one embodiment, the detainer 72 is detachably coupled to one of the receiver 48 and the face shield 34 such that the detainer 72 may be replaced, if desired. In one embodiment, one or more features (e.g., geometric properties) of the arm 70 or intermediate member 68 may be changed such that the force of resistance to rotation created by the detainer 72 is changed.

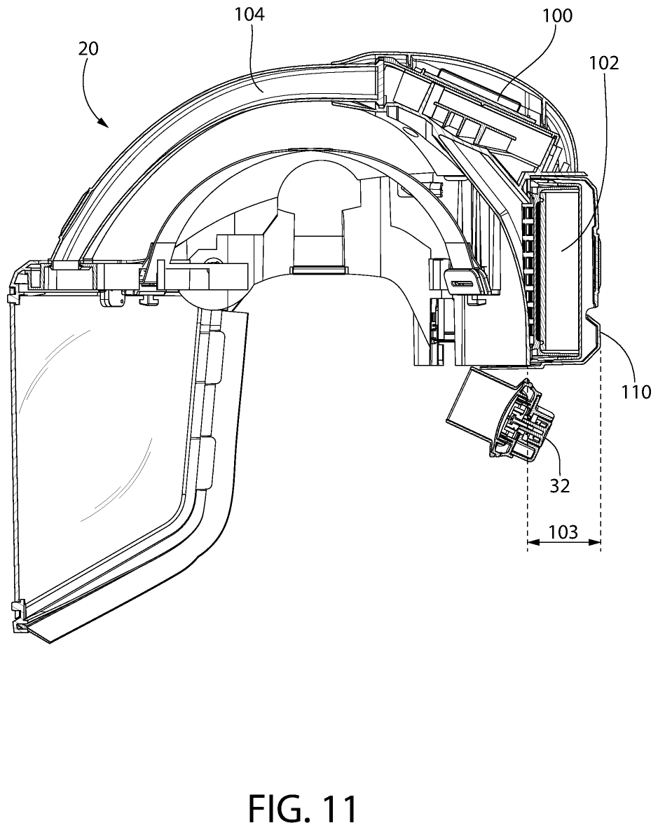

The protection system 20 may include a self-contained breathing system for filtering out hazardous material from the ambient air. Referring to FIGS. 4 and 11-12, the protection system 20 may include a blower 100 and a filter 102 (e.g., a PAPR system) detachably coupled to the frame 22 (FIG. 4). The weight of the current PAPR system may be reduced compared to existing head borne PAPR systems. In one embodiment, the PAPR system of the current design is about 40% lighter than existing PAPR systems. In one embodiment, the face shield 34 and the frame 22, which is capable of supporting a welding mask, weighs about 1.0 kg to about 1.5 kg, or about 1.3 kg. The frame 22 may include an air opening 106 (FIG. 6) such that air from the blower 100 can flow through a conduit 104 (FIG. 11) and into the space between the face shield 34 and the user's face such that a user has a supply of filtered air when the face shield 34 is in the lowered position. In one embodiment the blower 100 is configured to provide a continuous flow of air through the conduit 104. In one embodiment, the blower 100 is configured to provide pulses of air through the conduit 104. The blower 100 may be positioned atop a user's head when the protection system 20 is worn. In one embodiment, the blower 100 is above a user's ears when the protection system 20 is worn. The blower 100 may be positioned higher on the user's head than traditional PAPR systems. The blower 100 may be positioned above the halo 24 of the frame 22 (FIG. 4). In one embodiment, the center of gravity of the blower 100 and filter 102 is closer to the user's head than traditional head borne PAPR systems. A center of gravity closer to the user's head which may increase user comfort, reduce fatigue, and avoid unintentional contact between the PAPR system and external elements. In one embodiment, the center of gravity of at least one of the blower 100 and the filter 102 may be positioned between the front of the user's head and the back of the user's head. The filter 100 may wrap around a portion of a user's head to minimize the distance that the filter extends away from the frame 22 (e.g., rear overhang is minimized).

A diameter from the center of a user's head to an outer surface of the protection system 20 may be about 140 mm to about 180 mm, about 150 mm to about 170 mm, or about 160 mm. At least one of the filter 102 and the filter cover 110 may have an arcuate shape. A thickness 103 (FIG. 11) of the frame 22 and the filter cover 110 may be about 20 mm to about 40 mm.

Referring to FIG. 6, the frame 22 may include a receptacle 108 configured to receive the blower 100 (blower not shown in FIG. 6). The receptacle 108 may include a channel 110 to provide a fluid pathway between the receptacle and the conduit 104. The conduit 104, blower 100, and filter 104 may be detachably coupled to the frame such that the protection system 20 may be used with or without the PAPR system. In one embodiment, the receptacle 108 is angled relative to a user's head to minimize the distance that the filter extends away from the frame 22. In some embodiments, the channel 110 may have a relatively larger cross-sectional area to increase the volumetric flow of air from the blower to the space between the shield 34 and the user's face. In other embodiments, the channel 110 may have a relatively smaller cross-sectional area to increase the pressure of the flow of air from the blower to the space between the shield 34 and the user's face which may prevent external air from entering the space.

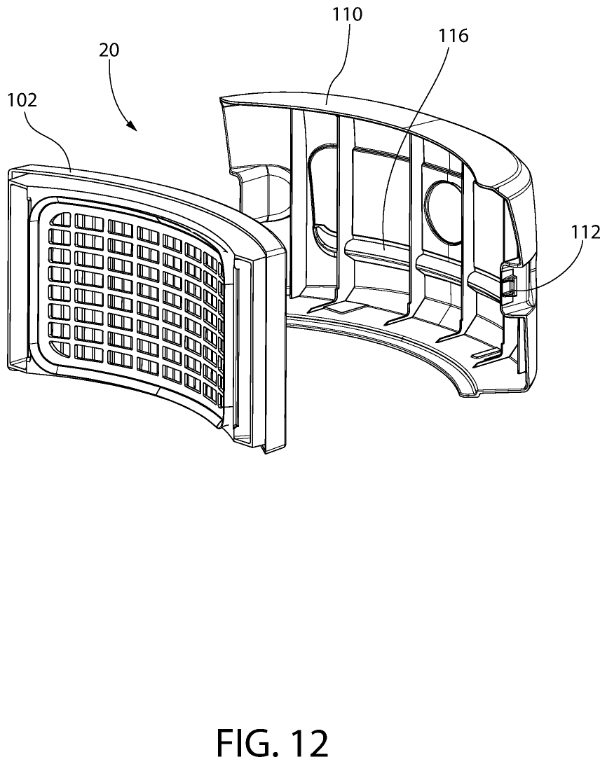

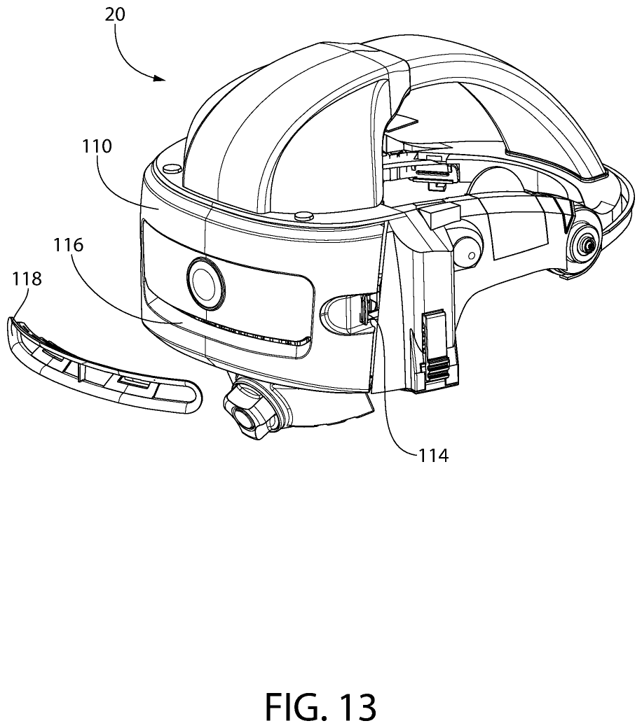

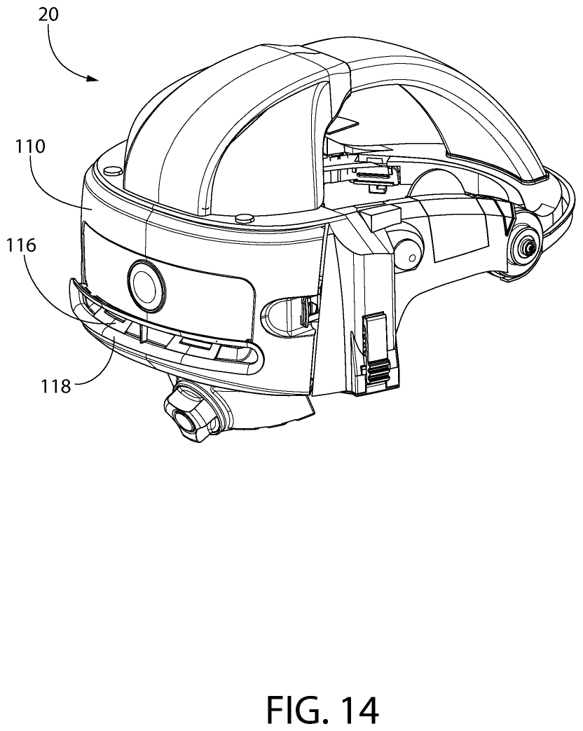

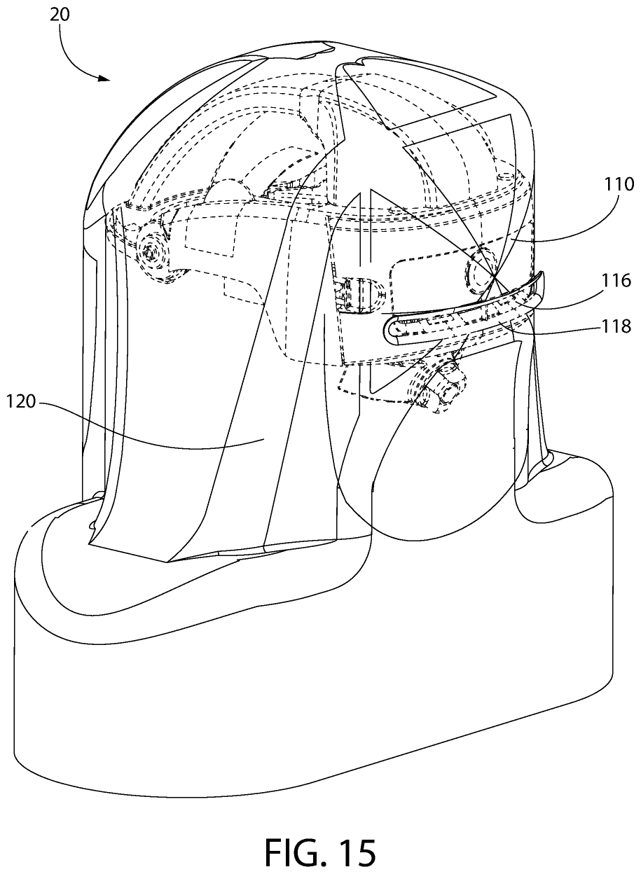

Referring to FIGS. 12-15, a filter cover 110 may be coupled to the frame 22. The filter cover 110 may be configured to receive the filter 102. The filter cover 110 may include a latch 112 (FIG. 12) configured to engage a deflectable arm 114 (FIG. 13) on the frame 22 to detachably couple the filter cover 110 to the frame 22. In one embodiment, the deflectable arm 114 is manually moveable such that the filter cover 110 can be decoupled/coupled to the frame 22, and the filter 102 can be replaced, without the use of any tools. The filter cover 110 may include an airway 116 configured to allow air to pass through the filter cover 110 such that the air can pass through the filter 102 and to the blower 100. In one embodiment, the airway 116 may have a surface area of about 25 cm² to about 50 cm², or about 38 cm². The protection system 20 may include an insert 118 (FIGS. 13-15) that can be positioned in the airway 116. The insert 118 may include an internal opening such that air can flow through the insert 118 when the insert 118 is within the airway 116. The insert 118 may be configured to couple to the filter cover 110 with a portion of a hood 120 (FIG. 15) between the insert 118 and the filter cover 110 such that an opening in the hood 120 is aligned with the airway 116 so the hood 120 does not occlude the airway 116 and air must pass through the

PAPR system before it is introduced to the user under the hood 120. In one embodiment, the hood 120 is used in manufacturing (e.g., pharmaceutical manufacturing). The hood 120 may be manufactured from a flexible material such that the hood 120 is a gasket that seals the space between the insert 118 and the filter cover 110. In one embodiment, the hood 120 is manufactured from a material comprising at least one of polyethylene and spunbonded polypropylene nonwoven composite.

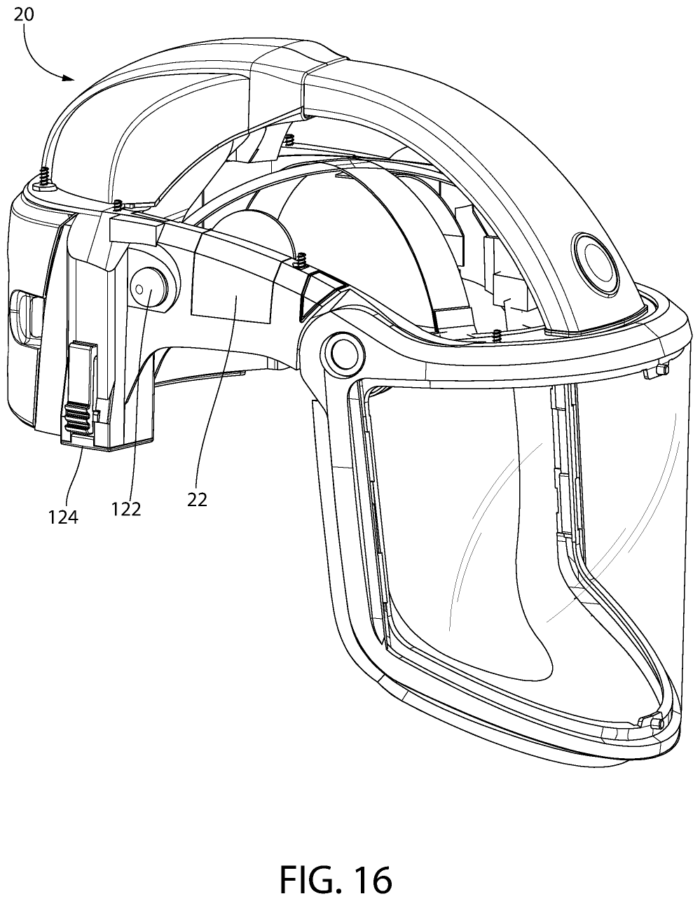

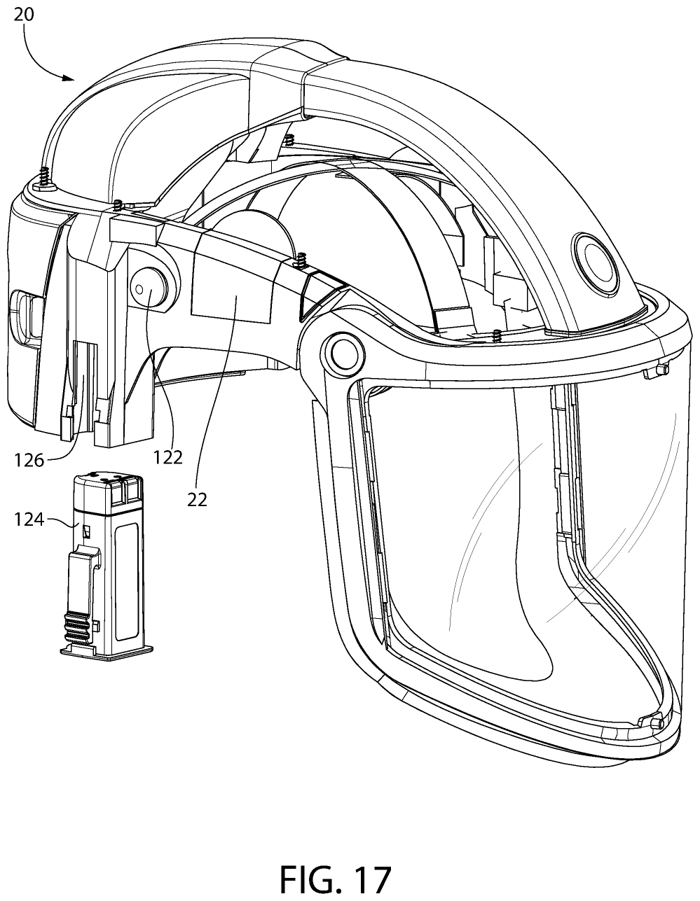

Referring to FIGS. 16-17, the blower 100 may be electrically connected to a power switch 122. The power switch 122 may be a pushbutton or toggle that can be activated with one hand. In one embodiment, the power switch 122 is configured to be activated with one hand and may be activated while wearing a glove. In one embodiment, the blower 100 is automatically turned on any time the face shield 24 is moved to the lowered position. A battery 124 may be detachably coupled to the frame 22. The battery 124 may provide power for the blower 100. The frame 22 may include a battery receiver 126. In one embodiment, the battery 124 can be detached from the battery receiver 126 with one hand while the protection system 20 is being worn by a user. In one embodiment, the battery 124 is slidably mounted in the battery receiver 126 through a bottom edge of the frame 22 and a release mechanism may face outwardly such that a user can engage the release mechanism to dismount the battery 124 from the battery receiver 126. In one embodiment, the release mechanism is a moveable, cantilevered beam that includes a lip which engages a ridge on the frame 22 to at least temporarily couple the battery 124 to the frame 22 and the user may engage (e.g., press with a finger) the cantilevered beam to decouple the battery 124 from the frame 22.

In some embodiments, the protection system 20 may be configured to provide a modular protection system wherein the frame 22 can be selectively coupled to any of a plurality of protection systems. In one embodiment, the frame 22 can be coupled to one or more of a face shield, a welding mask, a hood, ear muffs, and a PAPR system. A user may elect not to include some protection features or components and the protection system 20 may still provide the selected protection features without a degradation in performance of the other features. In one embodiment, a modular protection system 20 allows a user to utilize appropriate protection for different working environments.

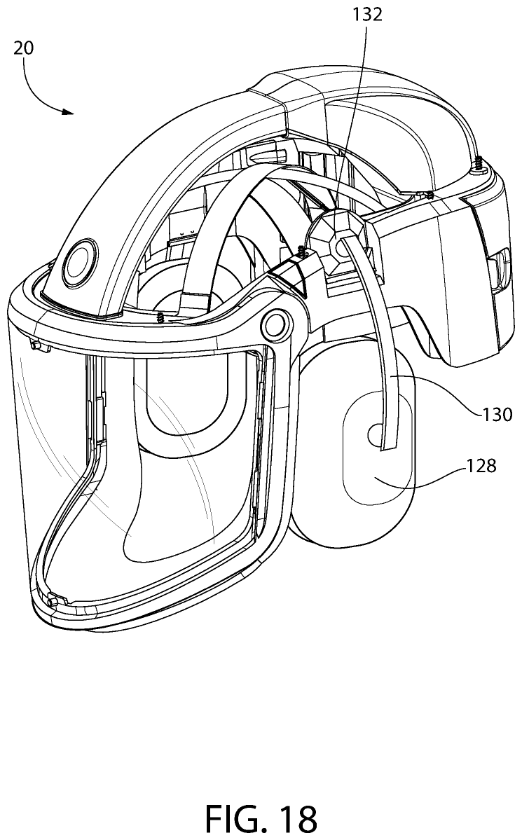

Referring to FIG. 18, the protection system 20 may include hearing protection. In one embodiment, the hearing protection is an ear plug configured to be inserted into a user's ear canal. In another embodiment, the hearing protection includes ear muffs 128. In another embodiment, the hearing protection includes ear muffs 128 and ear plugs. The ear muff 128 may be coupled to a strut 130. The strut 130 may include a tab 132 configured to be inserted into a slot 134 (FIG. 6) in the frame 22. The frame 22 may include a slot cover 136 (FIG. 5) which can cover the slot 134 when the ear muffs 128 are not coupled to the frame 22. The ear muffs 128 may be individually coupled to the frame 22 such that a user may selectively couple one or both ear muffs 128 to the frame 22. In one embodiment, the protection system 20 includes face protection and hearing protection without a hard hat.

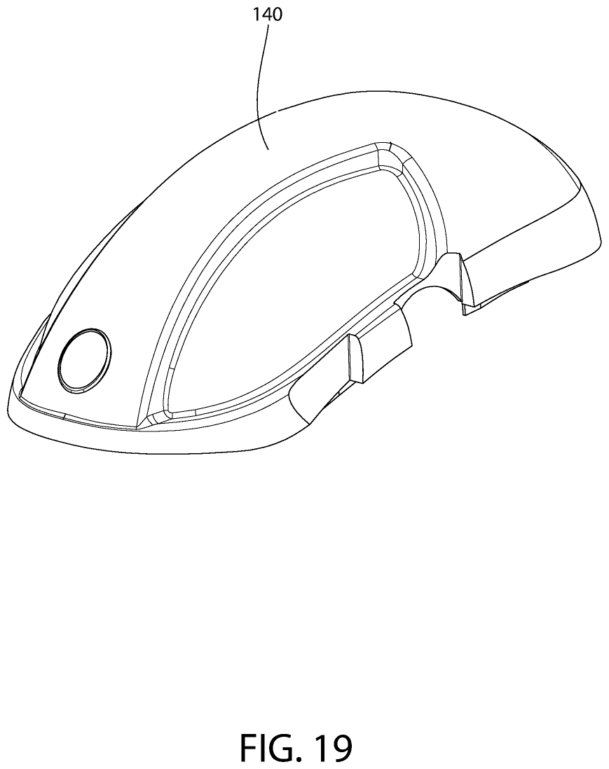

Referring to FIG. 19, a hard hat 140, or a portion of a hard hat, may be configured to be detachably coupled to the frame 22. The hard hat 140 may be coupled to the frame by snap fit, buckles, hook and loop fastener, snap fasteners, adhesive, magnets, or nuts and bolts. The hard hat 140 may provide protection for a user's head against external impacts. The hard hat 140 may also prevent fluid, dust, debris, and/or gasses from entering the space between the user's head and the frame 22. The hard hat thickness, or the materials used to manufacture the hard hat 140, may be selected based on the environmental hazards which will be encountered by a user. The frame 22 may define an opening configured to expose a user's head and the hard hat 140 may obscure the opening. The hard hat may provide protection from impact to the user's head to BS EN 397 and ANSI Z89.1 standards. The hard hat may be molded from a flame retardant material (e.g., ABS/Polycarbonate polymer blend).

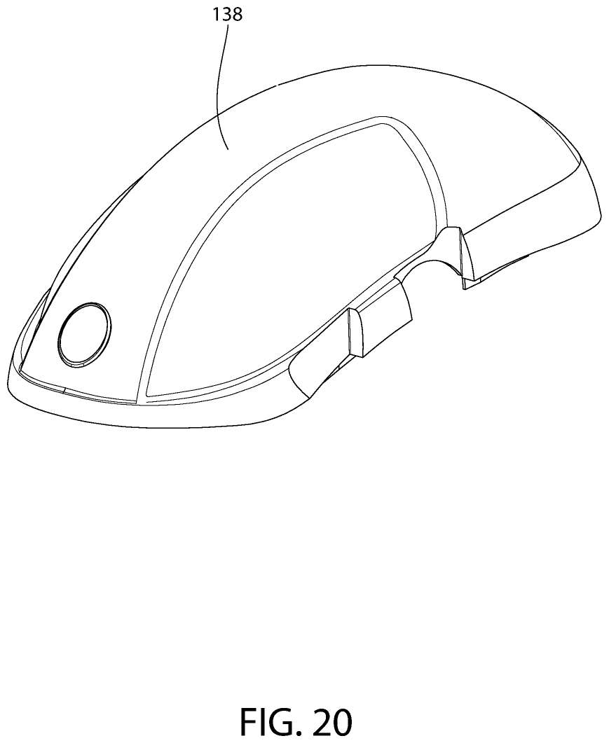

Referring to FIG. 20, a bump cap 138 may be coupled to the frame 22. The bump cap 138 may be a relatively light weight cover that provides protection against dust, debris, fumes, and/or fluid. The bump cap 138 may be similar to the hard hat 140 but the bump cap 138 may be of a relatively lighter weight than the hard hat 140. In some embodiments, the bump cap 138 may be about two thirds, about one half, about one third, or about one quarter of the weight of the hard hat 140. A light weight bump cap 138 may increase user comfort during use. In one embodiment, the bump cap 138 and the hard hat 140 may both be coupled to the frame 22 (e.g., the hard hat 140 may be configured to extend over the bump cap 138). In other embodiments, the bump cap 138 is removed from the frame 22 before attaching the hart hat 140 to the frame. In other embodiments, the bump cap 138 may be configured to couple over the hard hat 140 for ease of storage and/or additional protection. The bump cap 138, hard hat 140, and the frame 22 may provide two or more layers of protection for a user's head.

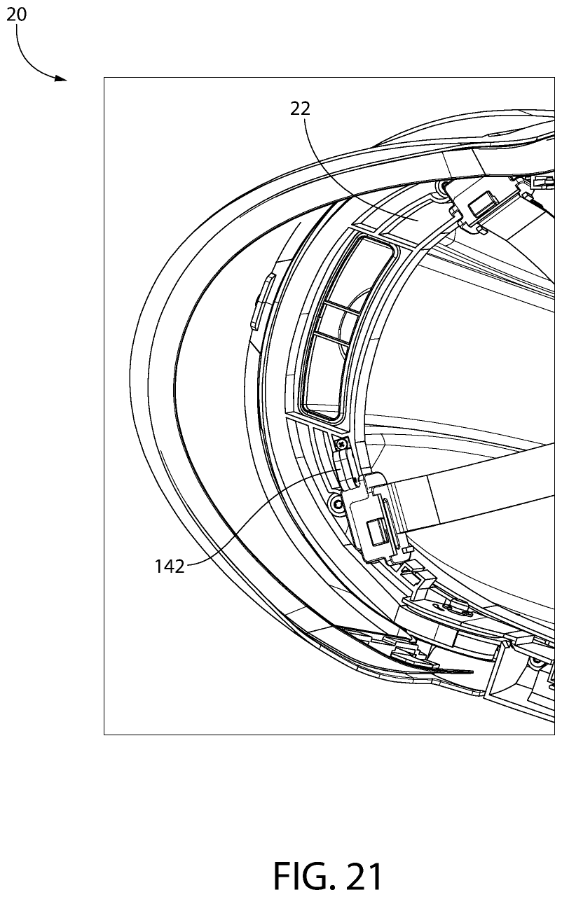

Referring to FIG. 21, the protection system 20 may include an indicator 142 to alert a user to a system status. In one embodiment, the indicator 142 is a light (e.g., an LED) that is in a user's line of sight such that the user can visually observe the indicator 142 while the protection system 20 is being worn. In another embodiment, the indicator 142 is an audio or tactile indicator that makes a sound or vibrates to provide an indication to the user. In one embodiment, the system status that causes the indicator 142 to be activated is that the battery 124 is running low or that the filter 102 needs to be changed. In one embodiment, the indicator 142 may not be in a user's direct line of sight but a user may observe a reflection of the indicator 142 on the face shield 34. In one embodiment, the indicator 142 may provide different indications for different statuses (e.g., a blinking light for low battery and a constant light for low air flow).

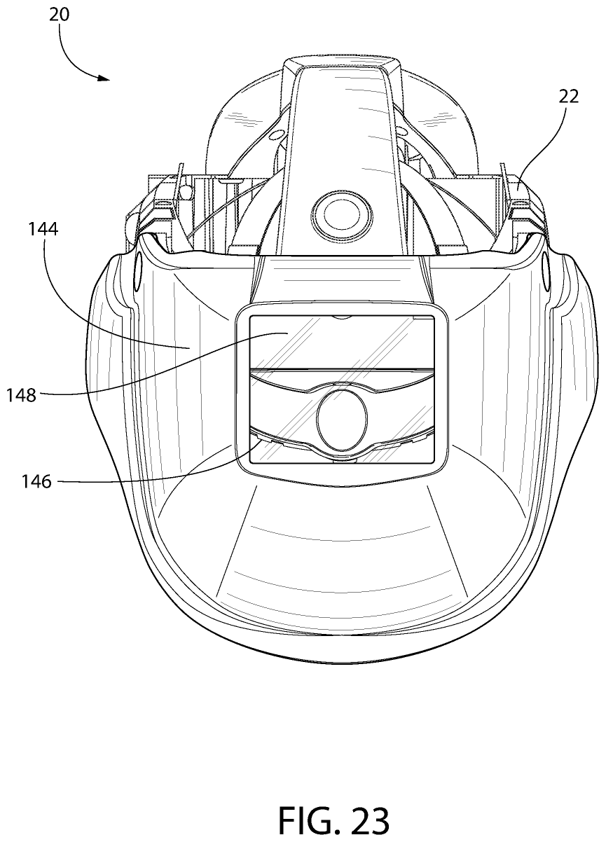

Referring to FIG. 23, a welding mask 144 may be coupled to the frame 22. The welding mask 144 may be moveably coupled to the frame 22 such that the welding mask 144 can be moved between a raised position and a lowered position as previously explained. A stiffer detainer 72 may be employed with the welding mask 144 than with the face shield 34 such that the coupling 46 can support the weight of the welding mask 144. The welding mask 144 may include a lens 146. The lens 146 may be an auto darkening lens. The welding mask 144 may include a sensor 148 (e.g., a light sensor) that senses a condition and sends a signal to an actuator configured to darken the auto darkening lens 146. In some embodiments, the auto darkening lens includes liquid crystal display technology (LCD) configured to block light (e.g., light emitted by a welding torch). The sensor 148 may be configured to send a signal to a controller or the LCD, thereby causing the LCD to darken.

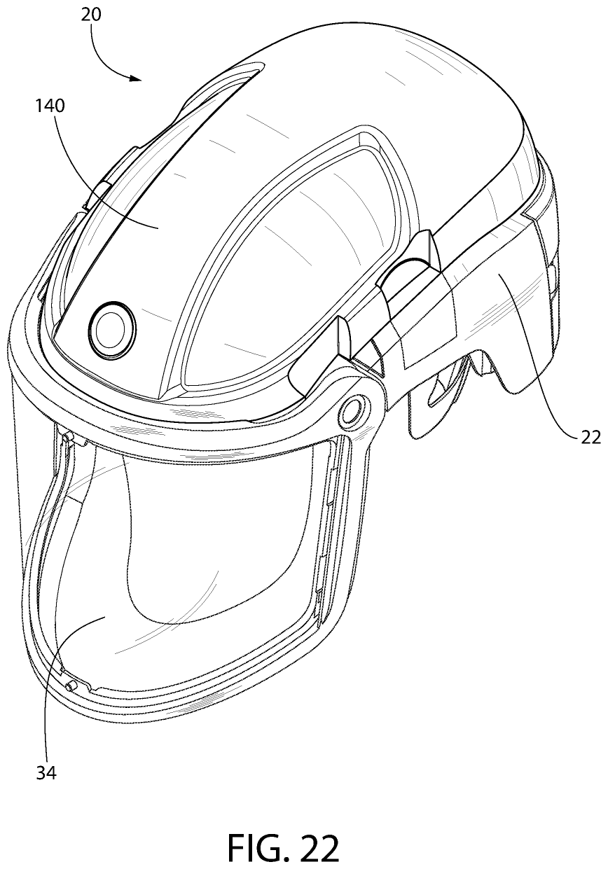

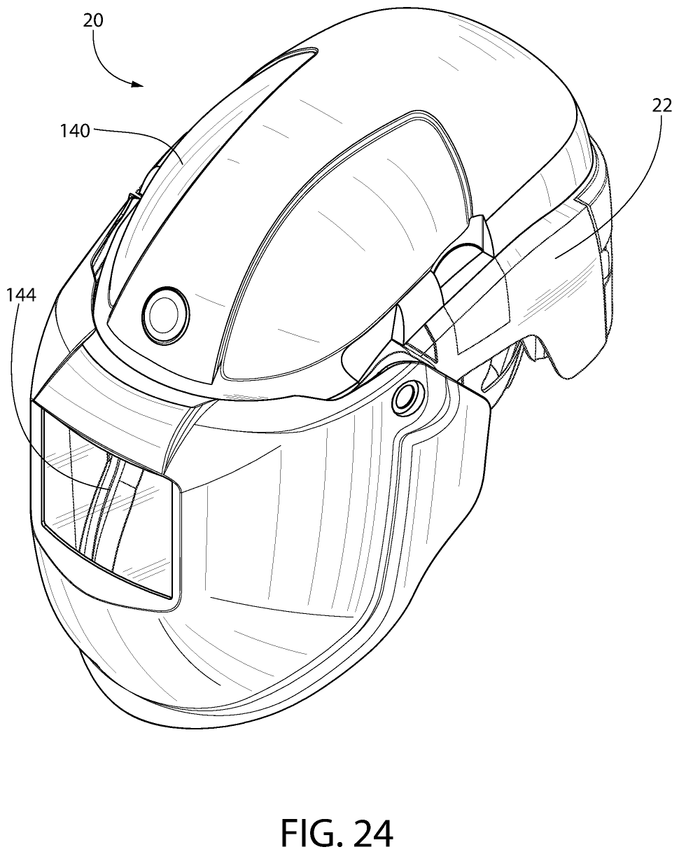

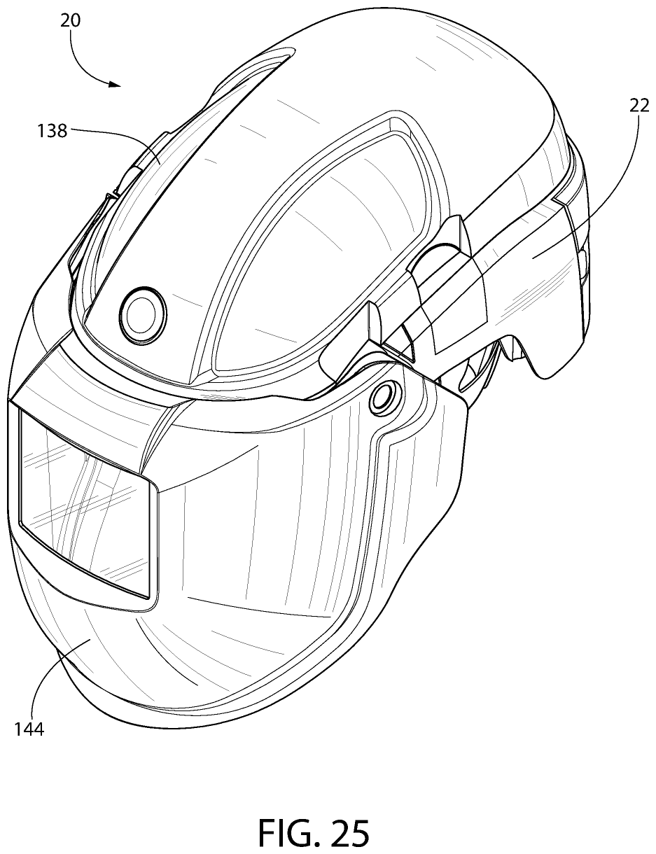

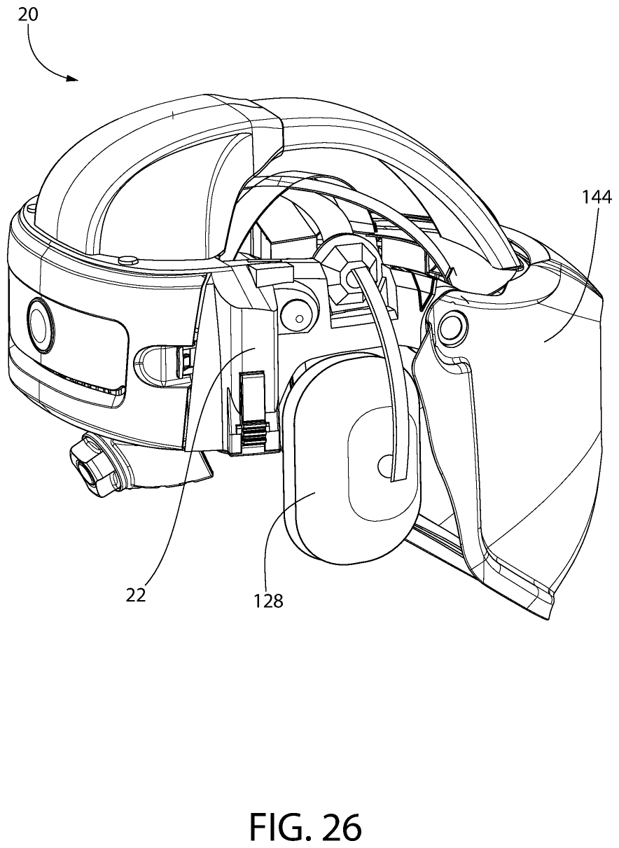

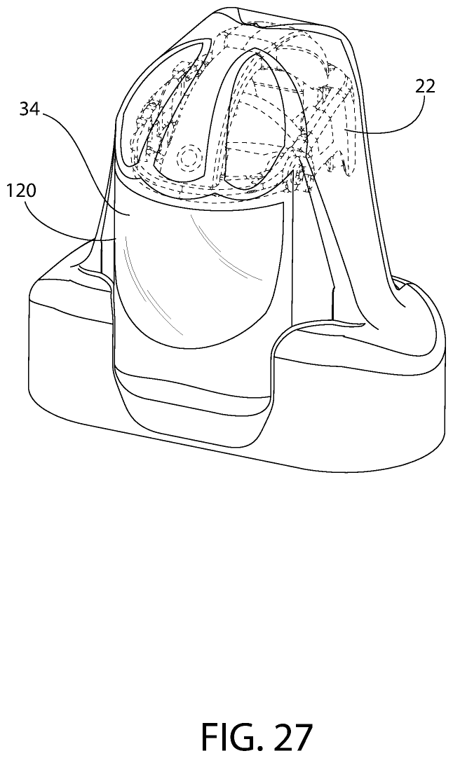

The protection system 20 may be modular such that the protection system 20 can be configured in a plurality of combinations. In one embodiment, the protection system 20 is provided as a kit that includes the frame 22 with the face shield 34 (FIG. 1). In another embodiment, the protection system 20 is provided as a kit that includes the frame 22 with the face shield 34 and the ear muffs 128. In another embodiment, the protection system 20 is provided as a kit that includes the frame 22 with the face shield 34 and the bump cap 138. In another embodiment, the protection system 20 is provided as a kit that includes the frame 22 with the face shield 34, the bump cap 138, and the ear muffs 128. In another embodiment, the protection system 20 is provided as a kit that includes the frame 22 with the hard hat 140 and the face shield 34 (FIG. 22). In another embodiment, the protection system 20 is provided as a kit that includes the frame 22 with the hard hat 140, the face shield 34, and the ear muffs 138. In another embodiment, the protection system 20 is provided as a kit that includes the frame 22 with the hood 120. In another embodiment, the protection system 20 is provided as a kit that includes the frame 22 with the hood 120 and the face shield 34 (FIG. 27). In another embodiment, the protection system 20 is provided as a kit that includes the frame 22 with the hood 120, the face shield 34, and the ear muffs 128. In another embodiment, the protection system 20 is provided as a kit that includes the frame 22 with the welding mask 144 (FIG. 23). In another embodiment, the protection system 20 is provided as a kit that includes the frame 22 with the welding mask 144 and the bump cap 138 (FIG. 24). In another embodiment, the protection system 20 is provided as a kit that includes the frame 22 with the welding mask 144 and the hard hat 140 (FIG. 25). In another embodiment, the protection system 20 is provided as a kit that includes the frame 22 with the welding mask 144 and the ear muffs 128 (FIG. 26). In another embodiment, the protection system 20 is provided as a kit that includes the frame 22 with the welding mask 144, the hard hat 140, and the ear muffs 128. In another embodiment, the protection system 20 is provided as a kit that includes the frame 22 with the welding mask 144, the bump cap 138, and the ear muffs 128. In another embodiment, the protection system 20 is provided as a kit that includes the frame 22 with the face shield 34 and the ear muffs 128 (FIG. 18).

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. The words “right”, “left”, “lower” and “upper” designate directions in the drawings to which reference is made. The words “inwardly” and “outwardly” refer to directions toward and away from, respectively, the geometric center of the protection system 20. Unless specifically set forth herein, the terms “a”, “an” and “the” are not limited to one element but instead should be read as meaning “at least one”.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

I/we claim:

1. A modular respiratory protection system comprising:
a frame defining an opening configured to expose a portion of a user's head;
an air filter;
a blower configured to draw air through the air filter and through a portion of the frame; and
a head protection element releasably coupled to the frame, the head protection element configured to cover the opening.

2. The modular respiratory protection system of claim 1, wherein the blower is configured to be above a user's ears when the frame is on the user's head.

3. The modular respiratory protection system of claim 1, further comprising:
a face shield moveably coupled to the frame.

4. The modular respiratory protection system of claim 3, further comprising:
a coupling configured to rotatably couple the face shield to the frame.

5. The modular respiratory protection system of claim 4, wherein the coupling is configured to at least temporarily maintain the face shield in a raised position.

6. The modular respiratory protection system of claim 4, wherein the coupling comprises a detainer and an intermediate member,
wherein the intermediate member is coupled to the face shield and is moveable relative to the detainer, and
wherein the detainer is configured to resiliently deflect as the intermediate member moves relative to the detainer.

7. The modular respiratory protection system of claim 6, wherein the intermediate member includes an arm and the detainer includes a detent,
wherein the detent is configured to at least temporarily prevent movement of the arm, and
wherein the arm is configured to cause the detainer to resiliently deflect as the face shield is moved between the raised position and the lowered position and the arm contacts the detent.

8. The modular respiratory protection system of claim 3, wherein the face shield is rotatable between a raised position and a lowered position and the blower is activated when the face shield is moved from the raised position to the lowered position.

9. The modular respiratory protection system of claim 1 further comprising:
an indicator configured to alert a user to a system status, wherein the indicator is within a user's line of sight when the frame is on the user's head.

10. The modular respiratory protection system of claim 1 further comprising:
a battery coupled to the frame, the battery being configured to supply power to the blower and the battery being configured to be decoupled from the frame while the frame is on the user's head.

11. The modular respiratory protection system of claim 10, wherein the battery is configured to be decoupled from the frame by the user with one hand.

12. The modular respiratory protection system of claim 1, wherein the frame is configured to be coupled to a face shield and ear muffs.

13. The modular respiratory protection system of claim 1, wherein the frame is configured to be selectively coupled to at least one of a face shield, ear muffs, and a welding mask.

14. The modular respiratory protection system of claim 1, wherein the frame is configured to be interchangeably coupled to a face shield and a welding mask.

15. The modular respiratory protection system of claim 14, wherein the welding mask includes an auto-darkening lens.

16. The protections system of claim 15, wherein the modular respiratory protection system includes a sensor configured to sense a condition and send a signal to an actuator configured to darken the auto-darkening lens.

17. The modular respiratory protection system of claim 1, wherein the frame is configured to be coupled to an ear muff

18. The modular respiratory protection system of claim 1, wherein the frame is configured to be coupled to a hood.

19. The modular respiratory protection system of claim 1, wherein the head protection element comprises a bump cap or a hard hat.

20. The modular respiratory protection system of claim 1, wherein the air filter is configured to wrap around a portion of the user's head.

21. A modular respiratory protection system comprising:
a frame defining an opening configured to expose a portion of a user's head;
a face shield moveably coupled to the frame;
a coupling configured to rotatably couple the face shield to the frame, the coupling including a detainer and an intermediate member, wherein the intermediate member is coupled to the face shield and is moveable relative to the detainer, and wherein at least a portion of the detainer is fixed relative to the frame and at least a portion of the detainer is configured to resiliently deflect as the intermediate member moves relative to the detainer;
an air filter;
a blower configured to draw air through the air filter and through a portion of the frame and deliver the air between the face shield and the user's head; and
a head protection element releasably coupled to the frame, the head protection element configured to cover the opening.

22. A modular respiratory protection system comprising:
a rigid frame defining an opening configured to expose a portion of a user's head;
a face shield moveably coupled to the frame;
an air filter configured to wrap around a portion of the user's head;
a blower configured to draw air through the air filter and through a portion of the frame, wherein the blower is configured to be above a user's ears when the frame is on the user's head;
a head protection element releasably coupled to the frame, the head protection element configured to cover the opening; and
a battery coupled to the frame, the battery being configured to supply power to the blower and the battery being configured to be decoupled from the frame while the frame is on the user's head.
